(19) United States
(12) Patent Application Publication
(10) Pub. No.: US 2003/0049325 A1
(43) Pub. Date: Mar. 13, 2003

(54) Agent for oral intake, its production and use

(76) Inventors: Wolfgang Suwelack, Billerbeck (DE); Petra Tewes-Schwarzer, Senden (DE); Rudiger Groning, Munster (DE)

Correspondence Address: Connolly Bove Lodge & Hutz LLP, 1220 Market Street, P.O. Box 2207, Wilmington, DE, 19899, US

(21) Appl. No.: 10/271,379

(22) Filed: Oct. 15, 2002

Related U.S. Application Data
(63) Continuation of application No. 09/086,030, filed on May 28, 1998.

(30) Foreign Application Priority Data
Sep. 5, 1997 (DE) 197 39 031.5

(51) Int. Cl. A61K 9/48; A61K 35/12

(52) U.S. Cl. 424/520

(57) ABSTRACT

The invention relates to an agent for oral intake containing at least one compressed, non-toxic carrier which is at least partially broken down, or can be broken down, or is eliminated or can be eliminated via the digestive tract, whereby the carrier, after expanding in the stomach, has a sponge-like structure, characterized in that the carrier, at least partially, has a collagen structure; it also relates to a process for the production of the agent of the above-mentioned type, wherein a fine-pore sponge consisting of collagen and having a density ranging from 0.005 g/cm³ to 1 g/cm³—which has optionally been treated with at least one active ingredient and/or additive prior to the pressing procedure, optionally in the presence of a mold-release agent—is compressed to half to one-fiftieth, preferably one-third to one-thirtieth of its original size, and it also relates to the use of this agent and of this process.

The present invention relates to an agent for oral intake which contains at least one compressed, non-toxic carrier that is at least partially broken down and/or eliminated via the digestive tract, whereby the carrier has a sponge-like structure after it has expanded in the stomach. Another subject matter of the present invention is a process for the production of the above-mentioned agent as well as the use of said agent for appetite control, as a nutritional supplement and also to administer cosmetics or pharmaceuticals.

The literature discloses several so-called pharmaceutical appetite inhibitors such as, for example, amphetamines which, by biochemical means, induce an aversion to food intake (lack of appetite). A number of chemically related compounds are also employed as appetite inhibitors. These are among the indirectly active sympathomimetics, and consequently also have an indirect glycogenolytic and lipolytic effect. Not all patients respond to appetite inhibitors and, even in cases of initial efficacy, this soon subsides. Moreover, since these agents usually have side effects, for instance, pulmonary hyper-tonia in the case of aminorex, as well as psychomotor stimulation and habit-formation all the way to addiction, they can only be used for special indications, for example, to initiate a diet treatment.

In addition to testing these pharmaceutically active appetite inhibitors, there have also been numerous experiments aimed at achieving a suitable inhibition of appetite with compressed, non-toxic carriers which are at least partially broken down and/or eliminated via the digestive tract, whereby such carriers expand in the stomach and create a feeling of fullness in the stomach and thus a sense of satiation.

EP-A-0,317,079 discloses an agent in which the components are placed into capsules practically as such, although in a compressed form, or else they are pressed into tablets. After being ingested, the capsule or the pressed tablet dissolves in the stomach, the components form a swollen mass that binds liquid present in the stomach and that is broken down or eliminated via the digestive tract. Since the agent does not have any caloric value but at the same gives the body a feeling of satiation due to the swollen mass present in the stomach, food intake can then be halted or reduced without great effort for purposes of weight loss. Since the swollen mass is essentially unbound, it passes relatively quickly from the stomach to the intestinal tract, as a result of which the feeling of satiation created only lasts for a relatively short period of time.

EP-A-0,043,317 discloses a hydrophilic substance on the basis of polyurethane-polyol which has the property of binding water and which is also meant for therapeutic and cosmetic applications. For the reasons mentioned above, however, this substance is not suited for weight-reduction purposes.

Furthermore, U.S. Pat. No. 4,401,682 as well as WO-90/01,879 disclose agents on the basis of cellulose fibers to which other ingredients have been added. These cellulose fibers plus the additional substances are comminuted into a fine powder and mixed with gelatin so that, upon being ingested, this agent likewise forms a gel-like mass with an undefined spatial structure in the stomach, and it can be broken down relatively quickly. This means that relatively large amounts of this agent have to be ingested at relatively short intervals in order to generate the desired feeling of satiation. This is unsuitable, both because of cost considerations and because of the greater risk of side effects associated with the increased mass throughput.

U.S. Pat. No. 4,735,214 relates to an agent for oral intake in a capsule made of gelatin that dissolves in water and releases the content, which is a non-toxic, low-calorie substance that increases in volume upon being released—in the case here, hydrophilic closed-cell polyurethane foam—whereby said substance consists of a body having a sponge-like structure that is placed into the capsule in a compressed form and retained in this form by the capsule. This agent is inserted into the stomach by means of a flexible cord and removed again after collecting a smear specimen; it serves as a capsule for conducting diagnostic procedures in the gastrointestinal tract, for instance, in order to detect stomach cancer.

Another agent that can be administered orally and that likewise contains compressed polyurethane with a gelatin capsule as the casing is the subject matter of U.S. Pat. No. 3,688,763. This capsule, however, also has an outer coating made of cellulose acetate phthalate so that the capsule does not dissolve in the stomach but rather only in the large intestine, where it releases the polyurethane sponge.

European patent application 0,202,159 describes a device consisting of at least one polymer that is soluble in the stomach and of at least one polymer that is not soluble in the stomach, whereby the insoluble polymer is selected from water-insoluble types of cellulose, among others.

According to FIG. 1, European patent application 0,344,939 relates to a system with a delayed action which remains in the stomach, whereby the core is made of a micro-porous polymer that loses its consistency as a result of dissolution, hydrolysis or enzymatic breakdown. This comprises, in particular, specially modified types of cellulose. The arms of this system can themselves consist of cellulose esters, among others.

U.S. Pat. No. 4,434,153 relates to a delayed-release preparation in which the active ingredient is embedded in a hydrophilic polymer, for example, a hydrogel of animal or vegetable origin or else a synthetic.

Patent Abstract of Japan C-742, Jul. 18, 1990, volume 14, no. 334, which is a summary of JP 2-121919 A, relates to a tablet that is produced by mixing 10% to 50% by weight of gelatin with 5% to 20% by weight of glycerin, 5% to 40% by weight of poly-acrylic acid as well as 0.03% to 60% by weight of a pharmaceutical agent by means of compression in a familiar manner, followed by a brief heat treatment. The tablet thus produced has good delayed-release properties in the digestive tract. This state of the art, however, does not provide any information about employing a carrier made of collagen and having a sponge-like structure.

EP-A 235,363 relates to a tablet with a delayed-release effect which contains a special active ingredient together with denatured egg albumin as well as preferably a coating agent, and which is obtained by means of dry-mixing of the two above-mentioned components, moistening and drying as well as tabletting, followed by a heat treatment at a temperature of at least 60° C. [140° F.]. This publication does not provide any information on a carrier made of collagen with a sponge-like structure.

DE-C 32 02 255 relates to a process for the production of a delayed-release preparation that is obtained in that a system consisting of one or more polypeptides, one or more proteins and one or more physiologically active substances is first ground up in the frozen state and mixed together mechanically and in that, in a subsequent step, the material is processed into a pressed blank. Preferably, this is followed by a treatment with electromagnetic radiation. In that publication, the term polypeptide refers to special polypeptides as defined in column 3, lines 20 through 31. Protein, in turn, refers to special structures as defined in column 3, lines 32 through 45. Moreover, it seems to be essential for this patent that an apparent film be formed by heating the polypeptides and proteins under pressure, in addition to which the subsequent irradiation with electromagnetic waves obviously has the objective of de-naturing the surface of the pressed blank, something which is not necessary according to the present invention.

EP-A-0,471,217 (and the partially corresponding DE-A 40 25 912, which serves as the basis for the priority) relates to an agent for oral intake with a casing that is soluble in the stomach and releases its contents; this casing is filled with a non-toxic, low calorie substance that increases in volume upon being released, and this substance can degrade inside the digestive tract or can be eliminated through it, whereby this substance is a sponge that is placed into the casing in a compressed form and retained in this form by the casing. Said low-calorie substance is preferably a cellulose sponge or a polyurethane foam. This above-mentioned method, however, is not very feasible in actual practice since the use of a polyurethane foam as a compressed, expandable substance is not permitted in Germany as well as in several other countries in accordance with the General Directive on Additives 89/107/EEC or the directives on other additives in the European Community. Moreover, the sponge cellulose or alveolar cellulose specifically mentioned for the first time in the subsequent application is not permitted, at least according to food product guidelines, since these guidelines allow exclusively microcrystalline cellulose and powder cellulose, methyl cellulose, hydroxypropyl cellulose, hydroxypropylmethyl cellulose, methylethyl cellulose and sodium carboxy methyl cellulose. Also, the agent described in the above-mentioned patent application is technically complex in that one of its indispensable components is a casing, that is to say, a hard gelatin capsule that is soluble in the stomach and that releases its contents.

The present invention has the objective of creating an agent for oral intake which has at least one compressed, non-toxic carrier that is at least partially broken down and/or eliminated via the digestive tract, whereby the carrier, after expanding in the stomach, has a sponge-like structure, of creating an agent that is technically easier to produce than the above-mentioned simple swelling masses or swelling masses in the form of a sponge, that stays in the stomach for a sufficiently long time and that is at least partially broken down in the digestive tract, and especially, that is approved for use in food products.

This objective is achieved by using a carrier that completely or partially has a special protein structure.

Thus, the present invention relates to an agent for oral intake which contains at least one compressed, non-toxic carrier that is at least partially broken down, or can be broken down, or is eliminated or can be eliminated via the digestive tract, whereby the carrier has a sponge-like structure after expanding in the stomach, characterized in that the carrier has, at least partially, a collagen structure and the carrier structure consists especially of collagen.

The collagen structures as employed according to the invention essentially comprise the so-called scleroproteins which are also known by the designation fiber proteins, stromal proteins or structural proteins and which constitute a group of water-insoluble, fibrous, animal proteins with a purely stromal or support function. The collagen is obtained from support tissue or connective tissue, skin, bone and cartilage.

In another preferred embodiment of the agent according to the invention, the carrier contains the amino acids glycine and hydroxyproline, preferably with the tripeptide sequence GlyXy, whereby X stands for any desired amino acid and hydroxyproline often appears instead of y.

According to another preferred embodiment, the carrier stems from the phylum Porifera, especially the class of Demospongiae. This is the zoological designation of the group of aquatic animals commonly referred to as sponges. These sea inhabitants have a shape that is without symmetry but organized in a polar manner as clusters, crusts, funnels and bowls, and as mushrooms and antlers, that is formed by a skeleton consisting of collagen-(spongin) fibers in which scleres of calcite or silicic acid are deposited. The sponges normally have three layers, of which the largest middle layer, namely, the mesohyl, consists of a gelatinous matrix containing collagen fibers. Please refer, for example, to “Lexikon der Biologie” [Encyclopedia of biology], volume 7, Freiburg, Germany 1986, under the entry “sponges” as well as op. cit. volume 8 under the entries “spongia”, “spongin”.

The phylum Porifera is divided into the classes Calcarea, that is to say, sponges with calcite deposits, Hexactinellida, in other words, those with special silicic acid deposits as well as Demospongiae, which encompasses those with a skeleton consisting of fiber or silicic acid.

The preferred class belongs to the group of the especially suitable class Demospongiae. These include, in particular, the horn siliceous sponge (Cornacu-spongia), the freshwater sponges and the bath sponge (Spongia orcinalis) with the subspecies Levantine sponge (Spongia officinalis mollissima), zimocca sponge (Spongia officinalis zimocca), elephant-ear sponge (Spongia officinalis lamella) as well as the horse sponge (Hippospongia communis) with its large holes.

The sponges harvested from the water are freed of mineral components in a known manner, for instance, by means of acidic digestion, so as to make it possible to isolate the collagen carrier as the fundamental component of the agent according to the invention.

According to another preferred embodiment, the carrier in the agent employed according to the invention is a collagen derived from natural animal matter. The production of these collagen fiber networks or collagen sponges—whose use is preferred—is a familiar process known, for example, from German laid-open application no. 18 11 290, German laid-open application no. 26 25 289, German patent no. 27 34 503 and especially from German laid-open application no. 32 03 957 of the applicant.

In accordance with another preferred embodiment, after the compressing procedure, the agent according to the invention has a sponge-like carrier with a density ranging from 0.005 g/cm³ to 1.0 g/cm³, preferably from 0.01 g/cm³ to 0.1 g/cm³. The density cited is measured according to German standard DIN 53 420.

In another preferred embodiment, the carrier in the agent according to the invention is not placed into a capsule, but rather, it is in the form of a pressed blank. In this context, we would like to refer to the monograph by Ms. Schöffling-Krause titled “Arzneimittel-formenlehre” [Treatise on drug delivery systems], Stuttgart, Germany 1987, pages 131 through 154 and to the production methods and machines described there as well as to the chapter titled “Tablets” in the book by Rudolf Voigt titled “Pharmazeutische Technologie für Studium und Beruf” [Pharmaceutical technology for students and professionals], published by Ullstein Mosby, Berlin, Germany 1993, page 205 ff. as well as to the production methods and machines described there. The material feed to the tablet presses is modified as a function of the material.

According to another preferred embodiment, the carrier in the agent according to the invention is in the form of a tablet. Once again, we would like to refer to the monograph by Schöffling-Krause. Depending on the production conditions, this tablet contains 0.001 grams to 5 grams, preferably 0.2 grams to 1 gram, relative to 100 grams of the agent, of at least one lubricant in the form of a (matrix) mold-release agent. Examples of this are siliconized talcum, cetyl talcum, magnesium stearate, PEG 4000-6000, stearic acid, cetyl alcohol, paraffin, beeswax, hydrated fats and oils and other physiologically tolerable mold-release agents. An overview of this can be found in the monograph by Rudolf Voigt in the chapter titled “Tablets”. Here, special preference is given to the use of an oblong tablet.

In another preferred embodiment, the tablet has a soluble coating surrounding the tablet. In this context, reference is made to the monograph by Schöffling-Krause, pages 144 through 148. This coating is normally applied in amounts of 0.1 grams to 50 grams, preferably from 1 gram to 20 grams, relative to 100 grams of the agent, and can consist, for instance, of film-forming coatings that are soluble in gastric juice such as, for example, a coating syrup on the basis of hydrogels or coating powders, color pigment suspensions, a smooth syrup or a hard wax solution or suspension. Film coatings with polymers that are resistance to saliva but that are soluble in gastric juice such as, for instance, poly-acrylates are also employed. Other film coatings are soluble cellulose derivatives such as hydroxypropyl cellulose. An overview of suitable film-forming substances is found, in turn, in the monograph by Voigt in the chapter titled “Dragées” on page 261 ff. Other suitable coatings are those produced according to the method used for making sugar dragées, as can be likewise seen in the chapter titled “Dragées” in the monograph by Voigt.

According to another preferred embodiment, the carrier in the agent according to the invention is encapsulated, that is to say, it is contained in a capsule that is soluble in gastric juice, for example, in the form of a soft-gelatin capsule, a gelatin hard-shell capsule or as a capsule with a modified release of the active ingredient. In this context, we would like to refer to the monograph by Schöffling-Krause titled “Arzneimittelformenlehre” [Treatise on drug delivery systems], Stuttgart, Germany 1987, pages 118 through 130 and to the production methods and machines described there as well as to the chapter titled “Capsules” in the book by Rudolf Voigt titled “Pharmazeutische Technologie für Studium und Beruf” [Pharmaceutical technology for students and professionals], published by Ullstein Mosby, Berlin, Germany 1993, and to the production methods and machines described there.

According to another preferred embodiment, the carrier of the agent according to the invention contains at least one active ingredient and/or additive. The active ingredients are added at various points in time during the manufacture of the sponge-like carrier materials. Examples of additives are approved colorants such as carotenoids or vitamins such as for instance, vitamin B₂. Active ingredients such as, for example, omeprazol can also be added at various points in time, for instance, prior to compressing the sponges.

According to another preferred embodiment of the agent according to the invention, the active ingredient is contained in a matrix, casing, bedding and/or another carrier material that controls the release. This effectuates the release of the active ingredient by means of membrane diffusion, pore diffusion, swelling, erosion, pore diffusion from the matrix, swelling with diffusion as well as swelling with disintegration. Here, reference is made to the monograph by R Voigt, the chapter titled “Peroral depot drug delivery system”. In particular, hydroxypropyl methyl cellulose is employed in this case as the carrier material that controls the release.

The present invention also has the objective of providing a process for the production of the above-mentioned agent.

Therefore, the invention also relates to a process for the production of the above-mentioned agent, characterized in that a fine-pore sponge consisting of collagen and having a density ranging from 0.0005 g/cm³ to 1.0 g/cm³—which has optionally been treated with at least one active ingredient and/or additive prior to the pressing procedure and optionally also with the use of a mold-release agent—is compressed to half to one-fiftieth, preferably one-third to one-thirtieth of its original size and optionally surrounded by a capsule that is soluble in gastric juice.

In another preferred embodiment of the process according to the invention, the fine-pore sponge is combined with a carrier layer for at least one active ingredient. In accordance with the production process for layered tablets, the carrier layer is compressed onto the pre-compacted sponge.

According to another preferred embodiment of the process according to the invention, the fine-pore sponge is treated with at least one active ingredient and/or additive before or during the pressing procedure, which consists at least of one step. This is preferably done in that the active ingredients and/or additives are applied in a familiar manner onto the carrier in the form of the sponge, for instance, either in pure form, dissolved in a solvent or else as a dispersion in the form of an emulsion or suspension.

The production and compression of the sponges are done, for example, after pre-compacting the sponge once it has been placed into an excentric press and using a compression tool with a lower and upper punch commonly employed for tablet production and with a suitable matrix (for instance, an oblong form, 1.8 cm×0.9 cm). With the punching, a pre-compacted sponge is compressed to form a tablet having a thickness of 4 mm. Active ingredients can also be incorporated into the collagen dispersion prior to the freeze-drying process.

Within the context of the present invention, a biologically active substance such as, for instance, a cosmetic or a pharmaceutical, can be employed as the active ingredient which, in particular, can be released during the time of residence in the stomach.

Examples of cosmetically active substances are vitamins, such as β-marotin or fatty acids which can systemically have a cosmetically advantageous effect on the skin in terms of a skin regeneration or skin tanning.

In addition to this, minerals and trace elements can also be employed as such active ingredients.

Another subject matter of the invention is an appetite-suppressing agent that contains the compressed, non-toxic carrier on a collagen basis according to the invention, or the use of the above-mentioned agent for purposes of appetite suppression.

This is preferably done in that nutritional supplements, particularly vitamins, minerals, fatty acids and/or dietary fiber are also added or incorporated into these carriers.

Examples of such nutritional supplements are vitamins, which are known to be divided into fat-soluble vitamins such as, for instance, retinol, retinoic acid, retinal, calciferol, that is to say, the D vitamins, the tocopherols or E vitamins and the K vitamins or phylloquinones. Vitamin A deficiency causes night blindness, vitamin D deficiency causes rickets and vitamin E deficiency increases the tendency towards oxidative hemolysis, causes hemolytic anemia, edema and increased irritability. Vitamin K deficiency impairs blood clotting and causes hemorrhaging.

Another group that can be employed according to the invention in the nutritional supplements includes water-soluble vitamins, such as vitamins of the B group, for example, vitamin B₁, thiamin, riboflavin, pyroxidine, nicotinic acid, corrinoids, folic acid and, as another group, ascorbic acid or vitamin C. Thiamin deficiency leads to beriberi, riboflavin deficiency can cause inflammation of the cornea and gives rise to increased vascularization. B₆-vitamin deficiency can cause seborrheic dermatitis, hypochromic anemia, peripheral neuritides as well as cerebral convulsions. There is an increased need for vitamin B₆ during pregnancy and following radiation therapy. A deficiency of nicotinic acid leads to pellagra, while a shortage of corrinoids causes pernicious anemia or even funicular myelosis. Deficiency of folic acid causes problems during pregnancy. Insufficient ascorbic acid leads to scurvy and to Moler-Barlow's disease.

The daily intake of vitamin by means of the agent according to the invention ensues, for example, from the recommendation for supplement intake as put forward by the German Society for Nutrition (DGE). Typical daily intakes of vitamins are also cited, for instance, in the monograph by Forth titled “Tharmakologie und Toxikologie” [Pharmacology and toxicology], 4^{th} edition, 1983, page 401.

Other typical components of the oral agent according to the invention used as a nutritional supplement can be minerals or trace elements which are to be supplied for prophylactic or therapeutic purposes. Examples of these are iron, zinc, copper, manganese, molybdenum, iodine, cobalt and selenium as essential elements for the human body. When it comes to the typical daily requirement, reference is made to the abovementioned monograph by Forth, the table on page 416.

In addition to the essential elements for the human body, in many cases it is also necessary to supplement calcium, which is not only needed for the bones and cell structure, but also for the entire metabolism of the body. The quantity of calcium normally obtained by the body from food is not always sufficient to meet the needs. Calcium provides bones and teeth with their strength.

Another essential element that can be supplied according to the invention is potassium, which plays an active role in the regulation of the osmotic pressure within the cells. Potassium is a component of the digestive tract of the stomach and intestines and is quickly resorbed.

Another essential component for nutritional supplementation is magnesium, which influences muscle function. Magnesium is an essential nutrient which is present in almost all cells and which controls the activation of enzymes involved in energy metabolism.

In addition, the agents according to the invention can also be employed to administer at least one, at least partially soluble, pharmacologically active substance, especially one with a local or systemic effect. This includes, for instance, pharmacologically active substances that act upon the central nervous system such as, for example, depressants, hypnotics, sedatives, tranquilizers, muscle relaxants, antiparkinsonian drugs, analgesics, antihypertensive drugs, chemotherapeutic agents, antiinflammatories, hormones, contraceptives, sympathomimetics, diuretics, antiparasitic agents, agents for the treatment of hyperglycemia, electrolytes, cardiovascular drugs.

Examples of water-soluble pharmaceuticals which can be delivered with a delayed release by the agent according to the invention include iron sulfate, aminocaproic acid, potassium chloride, mecamylamine hydrochloride, procaine hydrochloride, amphetamine sulfate, methamphetamine hydrochloride, phenmetrazine hydrochloride, bethanechol chloride, atropine sulfate, methascopolamine bromide, isoproparnidiodide, tridihexethyl chloride, oxoprenolone hydrochloride, metroprolone hydrochloride, cimetidine hydrochloride and the like.

Examples of pharmacologically active substances with limited solubility in water which can be released by the agent according to the invention are mecitine hydrochloride, phenoxy benzamine, thiethyl perazine maleate, anisindone, reserpine, acetolamide, methazol amide, chloropropamide, tolazimide, chloromadinone acetate, aspirin, progestin, corticosteroids, etc. When it comes to examples of medicinal drugs that can be released by the agent according to the invention, reference is made to the “Pharmazeutische Stoffliste” [Pharmaceutical substance list], 7^{th} edition, Frankfurt am Main, Germany, 1989. Typical examples of medicinal drugs that can be incorporated into such carriers are acyclovir, levodopa and riboflavin.

Finally, the present invention relates to the use of the above-mentioned agent for purposes of modified active-ingredient release.

The agent according to the invention contains, for example, 0.22 grams of the animal protein collagen per unit, corresponding to a mean physiological caloric value of approximately 4.0 kJ or about 1.0 kcal, in other words, 18 kJ or around 4.4 kcal per gram of the agent according to the invention.

The recommended daily dosage of the agent according to the invention is generally up to 10 units, preferably 6 to 9 units, three times per day, half an hour before meals.

The present invention will be explained in greater detail below by means of production and application examples and then compared with the state of the art. All parts cited below refer to parts by weight.

### PRODUCTION EXAMPLE 1 (PERORAL AGENT WITHOUT ACTIVE INGREDIENT)

A collagen sponge (length of 46 cm, width of 8 cm, thickness of 1.3 cm) weighing 12.8 grams is pre-compacted by means of a pneumatic press to a width of 1.5 cm, thus producing a strip (measuring 46 cm in length, 1.5 cm in width and 1.3 cm in thickness).

The initial material has the following characteristic data:

a water-absorption capacity >3000%;

a dry weight of 89%±5%;

an ash content <3%;

a pH value of 3.2±0.2 as well as

a density of 30 mg/cm³±10%.

The strip is placed in segments into an excentric press (tablet press EK 0, manufactured by the Korsch company of Berlin, Germany) and the material is punched out using a lower and upper punch as well as a matrix so as to form an oblong tablet (19 mm×8 mm), each with four notches on the top and bottom. The tablets have a thickness of 4 mm at a weight of approximately 400 mg. The tablets are dimensionally stable after the compressing operation. The pressed blanks expand in water at a temperature of 37° C. [98.6° F.] while absorbing water within a maximum of 5 minutes to form a sponge (1.9 cm×0.8 cm×8 cm).

The peroral agent thus obtained did not exhibit any volume increase even after being stored for at least 2 months in a humid atmosphere.

### PRODUCTION EXAMPLE 2 (PERORAL AGENT WITH MOLD-RELEASE AGENT)

A collagen sponge is pre-compacted to form a strip, as described in Production Example 1. The top and bottom of the strip are coated with the pulverulent mold-release agent magnesium stearate prior to the compressing operation. In the present example, 65 mg of magnesium stearate are used per strip. Each tablet contains approximately 2 mg of mold-release agent on its surface. As a result of the hydrophobic mold-release agent, the initial expansion of the sponge within the first minute is delayed. In water at a temperature of 37° C. [98.6° F.], the pressed blanks expand and absorb water within 5 minutes to form a sponge (1.9 cm×0.8 cm×8 cm).

The peroral agent thus obtained did not exhibit any volume increase even after being stored for at least 2 months in a humid atmosphere.

### PRODUCTION EXAMPLE 3 (PERORAL AGENT AS AN APPETITE INHIBITOR)

The production is carried out as in Production Example 1. A total of 5 grams of myristic acid, sodium myristate, ammonium myristate, triethanolamine myristate or other derivatives of fatty acids, relative to 100 grams of the agent, is added to the sponge. The peroral agent thus obtained can be employed as an appetite inhibitor.

The peroral agent thus obtained can be employed as an appetite inhibitor and does not exhibit any volume increase even after being stored for at least 2 months in a humid atmosphere.

### PRODUCTION EXAMPLE 4 (PERORAL NUTRITIONAL SUPPLEMENT)

Production Example 1 was repeated, whereby an additional 5 grams of natural vitamin E, relative to 100 grams of the agent, were added.

The nutritional supplement thus obtained did not exhibit any volume increase even after being stored for at least 2 months in a humid atmosphere.

### PRODUCTION EXAMPLE 5 (PERORAL COSMETIC)

Production Example 1 was repeated, except that 5 grams of the cosmetically effective substance β-carotin, relative to 100 grams of the agent, were also added.

The peroral cosmetic agent thus obtained did not exhibit any volume increase even after being stored for at least 2 months in a humid atmosphere.

### PRODUCTION EXAMPLE 6 (PERORAL AGENT WITH A PHARMACEUTICAL SUBSTANCE)

Production Example 1 was repeated, except that 20 grams of the drug levodopa (L-dioxyphenyl alanine), relative to 100 grams of the agent, were added to the fine-pore collagen sponge used as the initial material.

The peroral pharmaceutical agent thus obtained did not exhibit any volume increase even after being stored for at least 2 months in a humid atmosphere.

### PRODUCTION EXAMPLE 7 (PERORAL AGENT WITH A MODIFIED ACTIVE-INGREDIENT RELEASE)

Production Example 1 was repeated whereby, as a modified active-ingredient release system, the compressed collagen sponge is combined with another layer consisting of 200 mg of hydroxypropylmethyl cellulose containing approximately 100 mg of levodopa and 25 mg of benserazide (1-DL-serine-2-(2,3,4-trihydroxybenzyl) hydrazide) as the carrier for the depot drug. The release of the active ingredient takes place in vitro within 10 hours.

The peroral agent thus obtained did not exhibit any volume increase even after being stored for at least 2 months in a humid atmosphere.

### APPLICATION EXAMPLE 1 (IN VITRO)

The pressed blank described in Production Example 1 was tested at 37° C. [98.6° F.] for its degradability in the digestive tract by being placed in synthetic gastric juice according to USP XXMII with the addition of pepsin. This experiment indicated that the degradation of the collagen sponge started after 240 minutes. After the gastric juice was replaced with synthetic intestinal juice according to USP XXIII with the addition of pancreatin, the collagen sponges disintegrated completely after 5.5 hours. Also when placed into synthetic intestinal juice, the collagen dissolved completely within 6 to 8 hours.

### APPLICATION EXAMPLE 2 (IN VITRO)

The release of riboflavin (vitamin B₂) from a compressed collagen sponge according to Production Example 4 was studied in 0.01 N hydrochloric acid solution at 37° C. [98.6° F.]. Riboflavin was released over a period of time of more than 18 hours. After 1 hour, 3 mg of riboflavin had been released, this value was 5.5 mg after 2 hours, 7 mg after 3 hours, 8.2 mg after 4 hours, 9.2 mg after 5 hours, 12.9 mg after 10 hours and 16.4 mg after 15 hours.

### APPLICATION EXAMPLE 3 (IN VIVO STUDY OF THE STOMACH RESIDENCE TIME)

Two collagen sponges according to Production Example 4, which had been loaded with 10 grams of riboflavin, relative to 100 grams of the collagen sponge, were taken by 12 test subjects with 200 ml of tap water prior to eating or instead of a meal following overnight fasting. Standardized meal times were set as 30 minutes after intake as well as after 4 or 8 hours (composition: 1 whole-wheat roll, 10 grams of butter, 40 grams of cheese). The test subjects drank 200 grams of water every hour in order to ensure a sufficient volume of urine for the analysis of riboflavin.

Well-established, non-invasive examination methods commonly employed for pharmaceutical preparations were used to study the residence time of the collagen sponge in the stomach, whereby the time of residence of riboflavin in the stomach is ascertained indirectly on the basis of the time it takes for the renal clearance of riboflavin. Riboflavin is only absorbed by the body in the upper segment of the small intestine. Upon being released in the stomach, the dissolved riboflavin reaches the large intestine, where it is absorbed via an active absorption mechanism. The duration of the renal clearance of riboflavin is correlated with the residence time of the orally administered agent in the stomach. It was found that the renal clearance of riboflavin, that is to say, of more than 0.2 mg/h, takes place over an average time of 9.9 hours (arithmetic mean value).

### COMPARATIVE EXAMPLE 1 AND APPLICATION THEREOF (IN VIVO STUDY OF THE RESIDENCE TIME IN THE STOMACH WITH DEPOT TABLETS)

Instead of a collagen sponge according to the above-mentioned Production Example 4, a non-expanding riboflavin depot tablet (microtablet) with a diameter of 4 mm was studied. Each test subject received 2 tablets per day. It was found that the renal clearance of riboflavin, that is to say, of more than 0.2 mg/h, is only maintained over a period of 5.6 hours (arithmetic mean value), in other words, it is 4.3 hours shorter.

1. Agent for oral intake containing at least one compressed, non-toxic carrier that is at least partially broken down, or can be broken down, or is eliminated or can be eliminated via the digestive tract, whereby the carrier, after expanding in the stomach, has a sponge-like structure, characterized in that the carrier, at least partially, has a collagen structure.

2. Agent according to claim 1, characterized in that the carrier with the collagen structure contains the amino acids glycine and hydroxyproline.

3. Agent according to the preceding claims, characterized in that the carrier stems from the phylum Porifera, especially the class of Demospongiae.

4. Agent according to the preceding claims, characterized in that, prior to being compressed, the sponge-like carrier has a density ranging from 0.005 g/cm³ to 1 g/cm³, preferably 0.01 g/cm³ to 0.1 g/cm³.

5. Agent according to claim 1, characterized in that the carrier is not contained in a capsule.

6. Agent according to claim 1, characterized in that the carrier is in the form of a tablet, preferably an oblong tablet.

7. Agent according to claim 6, characterized in that the tablet has a soluble coating.

8. Agent according to the preceding claims, characterized in that the carrier also contains at least one active ingredient and/or additive.

9. Agent according to claim 9, characterized in that the active ingredient is contained in a matrix, casing, bedding and/or in another carrier material that controls the release.

10. Agent according to claims 1 through 4, 8 and/or 9, characterized in that the carrier is in a capsule.

11. Process for the production of the agent according to claims 1 through 10, characterized in that a fine-pore sponge consisting of collagen and having a density ranging from 0.005 g/cm³ to 1 g/cm³—which has optionally been treated with at least one active ingredient and/or additive prior to the pressing procedure and optionally also in the presence of a mold-release agent—is compressed to half to one-fiftieth, preferably one-third to one-thirtieth of its original size and optionally surrounded by a capsule that is soluble in gastric juice.

12. Process according to claim 11, characterized in that the pressing procedure is carried out in at least one step, preferably in at least two steps.

13. Process according to claim 11 or 12, characterized in that the fine-pore sponge is provided with a carrier layer for at least one active ingredient, in that the carrier layer is compressed onto the pre-compacted sponge in a manner known for the production of layered tablets.

14. Use of the agent according to claims 1 through 10, or which can be obtained according to claims 11 through 13, for purposes of appetite suppression.

15. Use of the agent according to claims 1 through 10, or which can be obtained according to claims 11 through 13, for purposes of nutritional supplementation, especially with vitamins, minerals, fatty acids and/or dietary fiber.

16. Use of the agent according to claims 1 through 10, or which can be obtained according to claims 11 through 13, for purposes of administering at least one cosmetically active substance, optionally in conjunction with at least one substance that heals or regenerates the skin.

17. Use of the agent according to claims 1 through 10, or which can be obtained according to claims 11 through 13, for purposes of administering at least one pharmacologically active substance that is at least partially soluble, especially having a local or systemic effect.

18. Use of the agent according to claims 1 through 10, or which can be obtained according to claims 11 through 13, for purposes of modified active-ingredient release.
